(19) United States
(12) Patent Application Publication
(10) Pub. No.: US 2024/0035976 A1
(43) Pub. Date: Feb. 1, 2024

(54) UV EXCITED MULTI SPECTRAL FLUORESCENCE BASED TISSUE ANALYSIS WITH RAMAN SPECTROSCOPY ZOOM-IN SCANNING

(71) Applicant: CytoVeris Inc., Famington, CT (US)

(72) Inventors: David Fournier, Northborough, MA (US); Rishikesh Pandey, Unionville, CT (US)

(21) Appl. No.: 17/920,640

(22) PCT Filed: Apr. 21, 2021
(86) PCT No.: PCT/US2021/028438

§ 371 Date: Oct. 21, 2022

(51) Int. Cl. G01N 21/64; G01N 21/65; G01N 33/483

(52) U.S. Cl. CPC G01N 21/6456; G01N 21/65; G01N 21/6402; G01N 33/4833; G01N 2201/06113; G01N 2201/062

(57) ABSTRACT

A method and system of analyzing a resected tissue specimen is provided. The method includes: a) using an imaging system to image a tissue sample with excitation light configured to produce fluorescent emissions from the tissue sample, the imaging producing signals representative of the fluorescent emissions from the sample; b) determining a presence or an absence of at least one suspect tissue region on the tissue sample; c) determining a spatial location of said at least one suspect tissue region determined to be present on the tissue sample; d) using the imaging system to image the suspect tissue region at the determined spatial location with excitation light configured to produce Raman scattering from the sample, the imaging producing signals representative of the Raman scattering from sample; and e) analyzing the determined suspect tissue region using the signals representative of the Raman scattering from the sample.

This application claims the benefit of U.S. Provisional Application No. 63/013,297, filed Apr. 21, 2020, which application is hereby incorporated by reference in its entirety.

### BACKGROUND OF THE INVENTION

### 1. Technical Field

The present disclosure relates to systems and methods for measuring, classifying tissue types and detecting tumor margin in excised tissue samples in general, and to systems and methods for detecting tumor margin using fluorescence and Raman spectroscopy in particular.

### 2. Background Information

For many decades, the reference method for the diagnosis of cancer has been histopathological examination of tissues using conventional microscopy. This process is known as Surgical Pathology. In Surgical Pathology, tissue samples may be produced from surgical procedures (tumor resection), diagnostic biopsies, or autopsies. Typically, these tissue samples go through a process that includes dissection, fixation, and cutting of the tissue sample into precisely thin slices which are stained for contrast and mounted onto glass slides. The slides are subsequently examined by a pathologist under a microscope, and the pathologist's interpretation of the tissue results in the pathology “read” of the sample.

Current surgical techniques to resect cancer are limited by the lack of a precise method to determine the boundary between normal cells and cancerous cells, known as the “tumor margin”, in real time during surgical procedures. As a result, the success of such surgical procedures relies on the experience and judgement of the surgeon to decide on how much tissue to remove around the tumor. As a result, surgeons often perform what is called cavity shaving, which can result in the removal of excessive amounts of healthy tissue. Conversely, many patients do not have the entire tumor removed during the initial surgery and may need a follow up surgery to remove residual cancer tissue. This can be traumatic to the cancer patient, adding stress, and resulting in long-term detrimental effects on the patient outcome.

Advanced optical and electromagnetic (EM) imaging approaches have been reported for the determination of tumor margin. These approaches include the use of fluorescence imaging [1-2], near-infrared spectroscopy [3], Raman spectroscopy [4], and terahertz reflectivity [5]. Additionally, the use of mass spectrometry to profile tumor/normal tissue boundaries has been reported [6]. In the latter methodology, a mass spectrometer may be coupled to a “pen” that allows testing of cancerous tissue by determination and differentiation of the metabolic products produced by cancer cells compared to normal tissue cells.

Fluorescence spectroscopy/imaging, in particular, has been proposed for tumor margin assessment. Fluorescence measurements require either the use of a fluorescent dye or an external contrast agent or rely on fluorophores intrinsic to the tissue matrix such as tryptophan, collagen, elastin, nicotinamide adenine dinucleotide (NADH), flavin adenine dinucleotide (FAD), porphyrins, or the like. A cancer-tissue responsive fluorescence agent can be applied to the surface of a tissue specimen to aid in distinguishing cancerous tissue versus normal, benign tissue. Such agents can be applied via a simple topical application; e.g., a solution containing the agent may be sprayed onto the tissue being examined.

Biomolecular changes occurring in the cell and tissue state during pathological processes and disease progression often result in alterations of the amount and distribution of endogenous fluorophores and characteristics of the tissue microenvironment. It is also well understood that tumor tissue, due to the marked differences in cell-cycle and metabolic activity can exhibit strong differentiated “intrinsic tissue” autofluorescence (AF) spectral characteristics. These changes can be used to identify/highlight regions of diseased tissue, such as cancer.

The micro-environment immediately adjacent cancer cells is typically acidic. The acidic environment may serve as a “biomarker” for cancer. The presence of a pH variation, and thus the presence of cancer tissue, can be visualized via pH-dependent dyes as agents, such as a seminaphtharhodafluor (“SNARF”) dye for example [7]. pH-dependent dyes such as SNARF can be immobilized to nanoparticles of various forms; e.g., polymers designed to bind generically to cell surfaces at the tissue surface. The fluorescence of these agents indicates the micro-environment pH of the local tissue. Further non-limiting examples of agents that may be used to mark cancerous tissue include: a) a polymer which can form a pH responsive nanoparticle which dissembles above a particular transition pH (sometimes referred to as a “pH Transistor” mechanism”) as described in U.S. Patent Publication No. 2018/0369424 by Gao et al, and the publication “A transistor-like pH nanoprobe for tumor detection and image-guided surgery”, T. Zhao et al., [8]; b) pH-tunable, highly activatable multicolored fluorescent nanoparticles, as described by Zhou et al. [9]; c) pH-wavelength dependent core/shell silica nanoparticles as described by Korzeniowska et al., [10]; and d) a fluorescence agent based on the modulation of the fluorescence lifetime of quantum dots by pH as described by Tan et al., [11]. These agents may be utilized individually or in various combinations.

While fluorescence spectroscopy/imaging has been utilized with varying degrees of success, due to broad and overlapping fluorescence spectral profiles of biomolecules the specificity of fluorescence spectroscopy/imaging is often limited. Raman spectroscopy, in contrast with fluorescence spectroscopy/imaging techniques, probes molecular vibrations for identification purposes. The molecular vibrational information encoded in Raman spectrum feature sharper peaks and offers unprecedented molecular finger-printing ability. Consequently, Raman spectroscopy can provide far greater sensitivities and specificities than is possible using fluorescence techniques. However, Raman spectroscopy is not a widefield technique and has a relatively slow measurement speed that hampers its practical utility within medical operative procedures; e.g., within a surgical time window.

What is needed is a system and method that is able to evaluate relatively large tissue regions in a desirably short period of time (e.g., real-time), and one that provides desirable sensitivity and specificity.

### SUMMARY

According to an aspect of the present disclosure, a method of analyzing a resected tissue sample is provided. The method includes: a) using an imaging system to image a resected tissue sample with excitation light configured to produce fluorescent emissions from the tissue sample, the imaging producing signals representative of the fluorescent emissions from the tissue sample; b) determining a presence or an absence of at least one suspect tissue region on the tissue sample; c) determining a spatial location of said at least one suspect tissue region determined to be present on the tissue sample; d) using the imaging system to image the determined suspect tissue region at the determined spatial location with excitation light configured to produce Raman scattering from the tissue sample, the imaging producing signals representative of the Raman scattering from tissue sample; and e) analyzing the determined suspect tissue region using the signals representative of the Raman scattering from the tissue sample.

In any of the aspects or embodiments described above and herein, the step of using the imaging system to image the resected tissue sample with excitation light configured to produce fluorescent emissions from the tissue sample may utilize a first excitation light source, and the step of using the imaging system to image the resected tissue sample with excitation light configured to produce Raman scattering from the tissue sample may utilize the first excitation light source.

In any of the aspects or embodiments described above and herein, the step of using the imaging system to image the resected tissue sample with excitation light to produce fluorescent emissions from the tissue sample and the step of using the imaging system to image the resected tissue sample with excitation light to produce Raman scattering from the tissue sample may include using a light detector to detect both the fluorescent emissions from the tissue sample and the Raman scattering from the tissue sample.

In any of the aspects or embodiments described above and herein, the step of using the imaging system to image the resected tissue sample with excitation light configured to produce Raman scattering from the tissue sample may utilize a Raman system to process the Raman scattering from the tissue prior to the light detector detecting the Raman scattering from the tissue sample.

In any of the aspects or embodiments described above and herein, the first excitation light source may be a laser, and may be a laser that produces said excitation light at about 265 nm.

In any of the aspects or embodiments described above and herein, the first excitation light source may be a time-of-flight camera, and the method may include producing a three-dimensional surface map of at least a portion of the resected tissue sample.

In any of the aspects or embodiments described above and herein, the step of using the imaging system to image the resected tissue sample with excitation light configured to produce fluorescent emissions from the tissue sample may utilize a first excitation light source, and the step of using the imaging system to image the resected tissue sample with excitation light configured to produce Raman scattering from the tissue sample may utilize a second excitation light source, and the first excitation light source and the second excitation light source may be different types of light source.

In any of the aspects or embodiments described above and herein, the first excitation light source may be an LED, and the second excitation light source may be a laser.

In any of the aspects or embodiments described above and herein, the step of using the imaging system to image the resected tissue sample with excitation light to produce fluorescent emissions from the tissue sample may utilize a first light detector to detect the fluorescent emissions from the tissue sample, and the step of using the imaging system to image the resected tissue sample with excitation light to produce Raman scattering from the tissue sample may utilize a second light detector to detect the Raman scattering from the tissue sample.

In any of the aspects or embodiments described above and herein, the step of using the imaging system to image the resected tissue sample with excitation light configured to produce Raman scattering from the tissue sample may utilize a spectrometer to process the Raman scattering from the tissue prior to the second light detector detecting the Raman scattering from the tissue sample.

According to another aspect of the present disclosure, a system for analyzing a resected tissue sample is provided that includes at least one excitation light source, at least one light detector, a Raman system, and a system controller. The at least one excitation light source is configured to produce excitation light. The system controller is in communication with the at least one light source, the at least one light detector, the Raman system, and a non-transitory memory storing instructions, which instructions when executed cause the controller to: a) control the at least one excitation light source to interrogate a resected tissue sample with the excitation light to produce fluorescent emissions from the tissue sample; b) control the at least one light detector to detect the fluorescence emissions from the tissue sample and produce first signals representative of the detected fluorescence emission; c) determine a presence or an absence of at least one suspect tissue region on the tissue sample using the first signals representative of the detected fluorescence emission; d) determine a spatial location of the at least one suspect tissue region determined to be present on the tissue sample using the first signals representative of the detected fluorescence emission; e) control the at least one excitation light source to interrogate the determined suspect tissue region at the determined spatial location with the excitation light to produce Raman scattering from the tissue sample; f) control the at least one light detector to detect the Raman scattering from the tissue sample and produce second signals representative of the detected Raman scattering; and g) analyze the determined suspect tissue region using the second signals representative of the Raman scattering from the tissue sample.

In any of the aspects or embodiments described above and herein, the instructions when executed may cause the controller to control the same at least one excitation light source to interrogate the resected tissue sample with the excitation light to produce the fluorescent emissions from the tissue sample, and to interrogate the resected tissue sample with the excitation light configured to produce the Raman scattering from the tissue sample.

In any of the aspects or embodiments described above and herein, the instructions when executed may cause the controller to control the same at least one light detector to detect the fluorescence emissions from the tissue sample and produce first signals representative of the detected fluorescence emission and to detect the Raman scattering from the tissue sample and produce second signals representative of the detected Raman scattering.

In any of the aspects or embodiments described above and herein, the instructions when executed may cause the controller to control the Raman system to process the Raman scattering from the tissue prior to the at least one light detector detecting the Raman scattering from the tissue sample.

In any of the aspects or embodiments described above and herein, the at least one excitation light source may be a laser and be a laser that produces said excitation light at about 265 nm.

In any of the aspects or embodiments described above and herein, the at least one excitation light source may include a first excitation light source and a second excitation light source, and the instructions when executed may cause the controller to control the first excitation light source to interrogate the resected tissue sample with excitation light to produce the fluorescent emissions from the tissue sample, and the instructions when executed may cause the controller to control the second excitation light source to interrogate the resected tissue sample with excitation light to produce said Raman scattering emissions from the tissue sample, and the first excitation light source and the second excitation light source may be different types of light source.

In any of the aspects or embodiments described above and herein, the first excitation light source may be an LED, and the second excitation light source may be a laser.

In any of the aspects or embodiments described above and herein, the at least one light detector may include a first light detector and a second light detector, and the instructions when executed may cause the controller to control the first light detector to detect the fluorescent emissions from the tissue sample, and the instructions when executed may cause the controller to control the second light detector to detect the Raman scattering emissions from the tissue sample.

In any of the aspects or embodiments described above and herein, the Raman system may include a spectrometer, and the instructions when executed may cause the controller to control the spectrometer to process the Raman scattering from the tissue prior to the second light detector detecting the Raman scattering from the tissue sample.

### BRIEF DESCRIPTION OF THE DRAWINGS

FIG. 1 is a diagrammatic representation of a system embodiment of the present disclosure.

FIG. 2 is a diagrammatic representation of a system embodiment of the present disclosure.

FIG. 3A is an image of an excised tissue sample shown in white light.

FIG. 3B is an image of the excised tissue sample shown in FIG. 2A, imaged using autofluorescence spectroscopy with a UV excitation wavelength.

FIG. 3C is an image of the excised tissue sample shown in FIG. 2A subjected to an H&E stain.

FIG. 4A is an image of an excised tissue sample shown in white light.

FIG. 4B is an image of the excised tissue sample shown in FIG. 3A, imaged using autofluorescence spectroscopy with a UV excitation wavelength.

FIG. 4C is an image of the excised tissue sample shown in FIG. 3A subjected to an H&E stain.

FIG. 5 is a diagrammatic representation of a system embodiment of the present disclosure.

### DETAILED DISCLOSURE

As will be described below, the present disclosure method and system 20 provide a means for determining the presence or absence of cancerous tissue (or other abnormal tissue), and therefore a means for evaluating tumor margin. The present disclosure system and method (which may be referred to as “RE-AFFIRM”, an acronym for Raman Encoded (Auto)fluorescence for Investigating Resection Margin) utilizes multi-spectral (auto)fluorescence imaging and Raman spectroscopy. The present disclosure is configured to gather multispectral imaging (MSI) autofluorescence data from a resected tissue sample (e.g., a suspected tumor body) to identify “suspect” tissue regions (i.e., regions of the tissue sample suspected of being cancerous or abnormal) for further investigation via Raman spectroscopy. The Raman spectroscopy with its greater sensitivity and specificity, in turn, is used to investigate those limited suspect tissue regions. Hence, the present disclosure provides the ability to determine the presence or absence of suspect tissue regions in an expedited manner, and to provide information with desirable sensitivity and specificity for those suspect tissue regions.

System embodiments of the present disclosure may include at least one excitation light source 22, at least one excitation light filter 24, at least one emission light filter 26, at least one light detector 28, a Raman system 30, and at least one system controller 32.

The excitation light source 22 may be an LED, a laser, or a filtered source of white light (e.g., flash lamps), or some combination thereof. The excitation light source 22 typically produces excitation light centered on a particular wavelength. The wavelength produced by the light source 22 is typically chosen based on the photometric properties associated with biospecies of interest; e.g., an excitation wavelength that produces a desirable fluorescence and Raman scattering emissions from one or more biospecies of interest. Non-limiting examples of wavelengths that the light source 22 may be configured to produce include wavelengths of about 265 nm, 280 nm, 340 nm, and 365 nm, as these wavelengths may be useful with respect to certain biospecies present within a tissue sample.

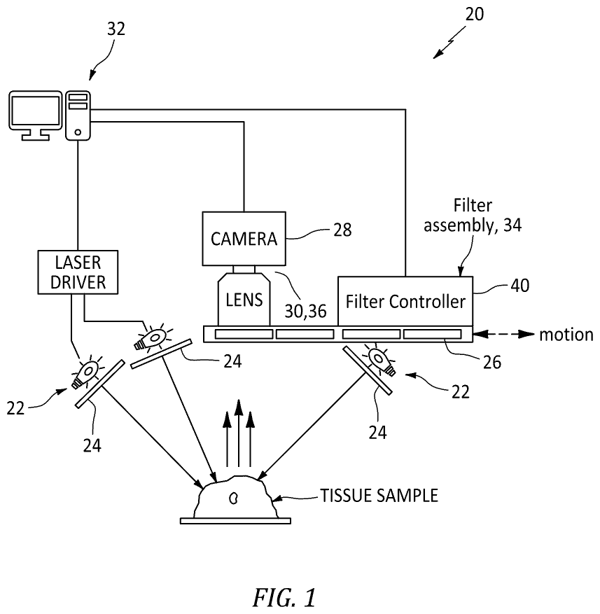

In some embodiments, the same excitation light source 22 may be used for both fluorescence imaging and Raman spectroscopy as those processes are described herein. An example of a system 20 that uses the same excitation light source 22 for both fluorescence imaging and Raman spectroscopy is described below and diagrammatically illustrated in FIG. 1. In these embodiments, the excitation light source 22 may be configured to produce excitation light within the sub-band of ultraviolet light between 200-280 nm (e.g., within the UVC sub-band of ultraviolet (UV) light, sometimes referred to as “deep ultraviolet light”). UV light at about 265 nm is particularly useful, as it produces high Raman scattering (e.g., 265 nm excitation produces 77× the amount of Raman scattering that is produced using 785 nm excitation) and a Raman spectrum that is substantially fluorescence free. In addition, excitation light at 265 nm excites almost all native tissue chromophores and therefore its use may avoid the need to excite using a plurality of different excitation wavelengths. A specific example of an acceptable light source 22 is a Nd-YAG laser configured to operate at a fourth harmonic to produce light at 266 nm.

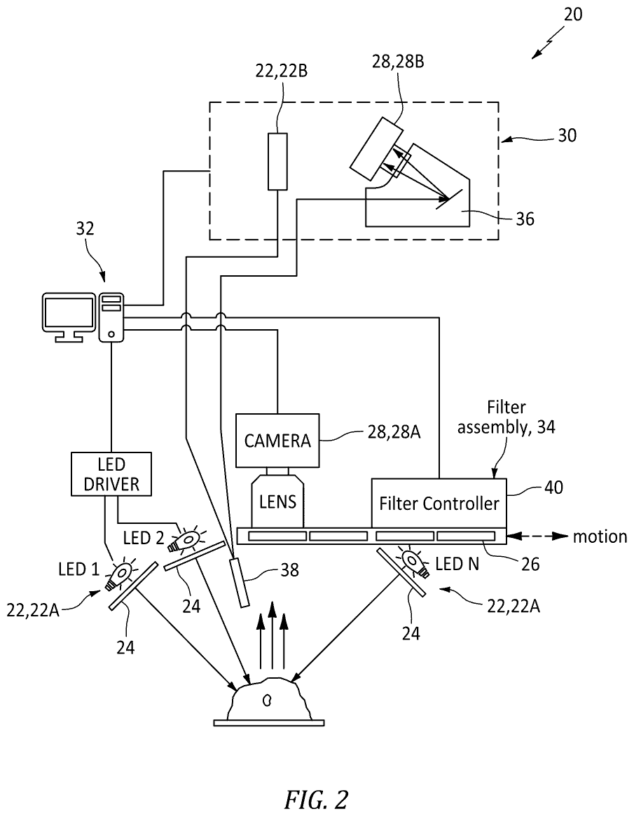

In some embodiments, a first excitation light source 22A (e.g., LEDs) may be used for fluorescence imaging and a second excitation light source 22B (e.g., lasers) may be used for Raman spectroscopy as those processes are described herein. An example of a system 20 that uses a first excitation light source 22A for fluorescence imaging and a second excitation light source 22B for Raman spectroscopy is described below and diagrammatically illustrated in FIG. 2.

In those embodiments that include one or more excitation light filters 24, the aforesaid light filters 24 may be configured to limit the bandwidth of the excitation light produced by the light source 22. The specific size of the bandwidth permitted by the excitation light filter 24 may be varied to suit the application. In some embodiments, the full width at half maximum (FWHM) of an excitation light filter 24 may be approximately 30 nm centered on the excitation wavelength. In some embodiments, the FWHM of an excitation light filter 24 may be approximately 10 nm centered on the excitation wavelength. For example, an excitation light filter 24 with a 30 nm bandwidth may be used for excitation wavelengths of 265 nm and 365 nm, and an excitation light filter 24 with a 10 nm bandwidth may be used for excitation wavelength of 340 nm. The present disclosure is not limited to these exemplary filter bandwidths and the exemplary wavelengths with which they are associated. The examination of different tissue types (e.g., breast tissue, brain tissue, etc.) may involve different tissue biospecies and therefore different excitation wavelengths and associated filter bandwidths. In some embodiments, an excitation filter 24 may be integral with an excitation light source 22. For example, an LED can be coated with specific formulation that allows only certain wavelengths or a predetermined range of wavelengths to be emitted from the excitation light source 22.

In those embodiments that include one or more emission light filters 26, the aforesaid emission light filters 26 may be configured to filter emitted light prior to the emitted light being received by the light detector 28. In those embodiments wherein the at least one emission light filter 26 includes a plurality of emission light filters 26, the plurality of emission light filters 26 may be disposed within a light filter assembly 34 that is configured to selectively position one of the respective emission light filters 26 in the light path between the light emitted from the tissue sample and the light detector 28; e.g., when a first light source is used to interrogate the tissue sample at a first wavelength, a first light emission light filter 26 may be positioned to receive light emitted as a result of the light interrogation, and when a second light source is used to interrogate the tissue sample at a second wavelength, a second light emission filter 26 may be positioned to receive light emitted as a result of the light interrogation, and so on. Light emission filters 26 having a band pass range of 385-400 nm, 400-420 nm, 420-450 nm, 450-550 nm, 600-650 nm are useful. These are exemplary emission light filter bandwidths, and the present disclosure is not limited thereto. In some embodiments, standard Bayer color filters and a “white” LED can be added to gather color pictures. In some embodiments, an emission light filter 26 may have a one pass range or may have a multiple pass range, e.g., a multi-band fluorescence bandpass filter. Additionally, wide-band optical spectral filters such as multivariate optical elements (MOEs) could also be utilized for fluorescence measurements.

In some embodiments, the same light detector 28 may be configured to detect both fluorescence emission and Raman scattered light from the interrogated tissue and produce signals representative thereof; e.g., see FIG. 1. In some embodiments, a first light detector 28A may be configured to detect fluorescence emission from the interrogated tissue and produce signals representative thereof and a second light detector 28B may be configured to detect Raman scattered light from the interrogated tissue and produce signals representative thereof; e.g., see FIG. 2. The signals produced by the light detector 28 are transferred to the system controller 32. Non-limiting examples of light detectors 28 include light detectors that convert light energy into an electrical signal such as a simple photodiode, or other optical detectors of the type known in the art, such as CCD arrays, CMOS, ICCD, etc. FIGS. 1 and 2 illustrate a light detector 28 in the form of a camera, which may be a monochrome or color camera. In some embodiments, optical fibers or fiber bundles may be used to convey light to the light detector 28. In some embodiments, the light detector 28 may be configured to specifically detect certain wavelengths of emitted light. Non-limiting examples of such a detection system include an RGB camera, a hyperspectral camera, and the like.

The Raman system 30 is configured to process emitted light signals resulting from Raman scattering. The Raman system 30 may include a traditional commercial spectrometer 36 or include optical elements such as a dispersion grating and a light detector. In some embodiments Raman measurement could be achieved within the Raman system 30 without a dispersing element or in a spectrometer-free manner using sets of optical filters. Also, different variants of Raman spectroscopy such as SRS, CARS, SERS can be employed. The Raman system 30 is not limited to any wavelength of excitation and the spectral acquisition/investigation is not limited to a specific wavenumber region. The system 20 embodiment shown in FIG. 2 includes a Raman system 30 as a component of the present disclosure system. In this system 20 embodiment, the Raman system 30 includes a Raman probe 38 that accepts light from a laser light source 22B and produces that light for interrogation of the tissue body. The Raman probe 38 is also configured to accept emitted light caused by Raman scattering and transfer that emitted light to a spectrometer 36, which in turn passes the emitted light to a Raman scattering light detector 28B (e.g., a camera). The Raman scattering light detector 28B, in turn, provides signals representative of the emitted light to the system controller 32. In this system embodiment, as stated above, the excitation light source 22 includes a first excitation light source 22A (e.g., LEDs) for fluorescence imaging and a second excitation light source 22B (e.g., lasers) for Raman spectroscopy. Also in this embodiment, the at least one light detector includes a first light detector 28A to detect fluorescence emission from the interrogated tissue and a second light detector 28B (e.g., within the Raman system) to detect Raman scattered light. The system 20 embodiment shown in FIG. 1 includes a Raman system 30 as a component. In this system 20 embodiment, the excitation light source 22 is used for both fluorescence imaging and for Raman spectroscopy. Also in this embodiment, the light detector 28 is configured to detect both fluorescence emission and Raman scattered light. In this embodiment, a spectrometer 36 or optical elements (e.g., dispersion grating and/or optical filters) may be used in combination with the light detector 28 to process the Raman scattering light. A distinct advantage of the present disclosure stems from its ability to fluorescently image and Raman scattering image the same excised tissue body without moving the tissue body. This ability avoids spatial registration errors that may otherwise occur and greatly expedites the process.

The system controller 32 is in communication with other components within the system, such as the light source 22, the light detector 28, the Raman system 30, and the like to control and/or receive signals therefrom to perform the functions described herein. The system controller 32 may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system to accomplish the same algorithmically and/or coordination of system components. The system controller 32 may include a single memory device or a plurality of memory devices. The present disclosure is not limited to any particular type of non-transitory memory device, and may include read-only memory, random access memory, volatile memory, non-volatile memory, static memory, dynamic memory, flash memory, cache memory, and/or any device that stores digital information. The system controller 32 may include, or may be in communication with, an input device that enables a user to enter data and/or instructions, and may include, or be in communication with, an output device configured, for example to display information (e.g., a visual display or a printer), or to transfer data, etc. Communications between the system controller 32 and other system components may be via a hardwire connection or via a wireless connection.

FIG. 2 diagrammatically illustrates a present disclosure system 20 that includes a light source having a plurality of LEDs and an LED driver, a plurality of excitation light filters 24, an emission light filter assembly 34 having a plurality of emission light filters 26 and a filter assembly controller 40, a light detector 28A having a camera, a Raman system 30, and a system controller 32 with an output device and an input device. The system 20 is shown diagrammatically in use with a tissue sample. The light source 22A is shown as having “N” number of LEDs, where “N” is an integer equal or greater than two. The system 20 is configured such that the LEDs produce excitation light from a plurality of different orientations so that substantially all the tissue sample may be interrogated with excitation light at various angles. The LED driver is in communication with the system controller 32 for selective operation of the respective LEDs. The emission light filter assembly 34 is configured to selectively position one of the respective emission light filters 26 in the light path between the light emitted from the tissue sample and the light detector 28A. The emission light filter assembly 34 is diagrammatically shown as having a linear array of emission light filters 26, but the present disclosure is not limited this configuration. The filter assembly controller 40 is in communication with the system controller 32 for selective operation of the emission light filter assembly 34. The Raman system 30 includes a Raman probe 38 that accepts light from a laser light source 22A and produces that light for Raman interrogation of the tissue body. The Raman probe 38 is also configured to accept emitted light caused by Raman scattering and transfer that emitted light to a spectrometer 36 (or other optical hardware as described herein), which in turn passes the emitted light to a light detector 28B (e.g., a camera). The light detector 28B, in turn, provides signals representative of the emitted light to the system controller 32. In this system embodiment, a first excitation light source 22A (e.g., LEDs) is used for fluorescence imaging and a second excitation light source 22B (e.g., lasers) for Raman spectroscopy. Also in this embodiment, a first light detector 28A is used to detect fluorescence emission from the interrogated tissue specimen and a second light detector 28B (e.g., within the Raman system 30) is used to detect Raman scattered light.

An example of the system shown in FIG. 2 in operation may be described as follows. An excised tissue specimen (e.g., a body of tissue understood to contain cancerous tissue, or abnormal tissue) is positioned to be interrogated by a first light source 22A and a second light source 22B. The system controller 32 is configured to operate the LEDs of the first light source 22A in the manner described herein to produce fluorescence emission. The first light source 22A includes a plurality of devices (e.g., LEDs) at orientations such that all surface portions of interest of the tissue specimen may be interrogated with excitation light. For a given tissue specimen interrogation, all the LEDs are configured to produce excitation interrogating light at the same wavelength. The present disclosure is not limited to fluorescence excitation light interrogation at any particular wavelength. In some embodiments, the first light source 22A may be configured so that the tissue specimen may be interrogated at a first wavelength, and subsequently at a second wavelength, etc., to facilitate different tissue biospecies fluorescence response. Also in this embodiment, the system controller 32 is configured to operate the laser(s) 22B of the Raman system (e.g., the second light source) in the manner described herein to produce Raman scattering. In this embodiment, the system 20 may be configured so that an operator can manually use the Raman probe 38 to examine suspect tissue regions (e.g., the operator may guide the probe 38 based on a visual depiction of the tissue specimen showing the suspect areas identified from the fluorescence imaging), or the system 20 may be configured so that the Raman probe 38 is automatically guided to the suspect tissue regions, or any combination thereof.

As described above, an excitation light filter 24 may be disposed in the light path between the respective LED 22A (or other type light generating device) and the tissue sample so that the produced fluorescence excitation light must pass through the respective filter 24 prior to impingement on the tissue sample. Each respective excitation light filter 24 is configured to limit the bandwidth of the excitation light produced by the light source 22A.

The tissue interrogating fluorescence excitation light is configured to produce a fluorescent emission response from the interrogated tissue. In some applications, the present disclosure may rely upon intrinsic fluorescence of the tissue specimen, e.g., an intrinsic fluorescent response to the excitation light from tissue biospecies (i.e., fluorophores) such as tryptophan, collagen, elastin, nicotinamide adenine dinucleotide (“NADH”), flavin adenine dinucleotide (“FAD”), porphyrins, etc. Biomolecular changes occurring in the cell and tissue state during pathological processes and disease progression often result in alterations of the amount and distribution of endogenous fluorophores and characteristics of the tissue microenvironment. It is also well understood that tumor tissue, due to the marked differences in cell-cycle and metabolic activity, can exhibit strong differentiated “intrinsic tissue” autofluorescence (AF) spectral characteristics. In other applications, the present disclosure may utilize one or more extrinsic agents to facilitate a useful fluorescent response. For example, a cancer-tissue responsive fluorescence agent can be applied to the surface of a tissue specimen to aid in distinguishing cancerous tissue versus normal, benign tissue. Such agents can be applied via a simple topical application; e.g., via a solution containing the agent may be sprayed onto the tissue being examined.

The micro-environment immediately adjacent cancer cells is typically acidic. The acidic environment may serve as a “biomarker” for cancer. The presence of a pH variation, and thus the presence of cancer tissue, can be visualized via pH-dependent dyes as agents, such as a seminaphtharhodafluor (“SNARF”) dye for example [7]. pH-dependent dyes such as SNARF can be immobilized to nanoparticles of various forms; e.g., polymers designed to bind generically to cell surfaces at the tissue surface. The fluorescence of these agents indicates the micro-environment pH of the local tissue. Further non-limiting examples of agents that may be used to mark cancerous tissue include: a) a polymer which can form a pH responsive nanoparticle which dissembles above a particular transition pH (sometimes referred to as a “pH Transistor“mechanism”) as described in U.S. Patent Publication No. 2018/0369424 by Gao et al, and the publication “A transistor-like pH nanoprobe for tumor detection and image-guided surgery”, T. Zhao et al., [8]; b) pH-tunable, highly activatable multicolored fluorescent nanoparticles, as described by Zhou et al. [9]; c) pH-wavelength dependent core/shell silica nanoparticles as described by Korzeniowska et al., [10]; and d) a fluorescence agent based on the modulation of the fluorescence lifetime of quantum dots by pH as described by Tan et al., [11]. These agents may be utilized individually or in various combinations. The present disclosure is not limited to these agent examples.

Regardless of whether the fluorescent emissions are a product of intrinsic tissue fluorescence or associated with an extrinsic agent that is configured to locate cancerous tissue via fluorescent emissions, the fluorescent emissions result from the excitation light and may be detected by the light detector 28A. Prior to the fluorescent emissions being detected by the light detector 28A, the aforesaid fluorescent emissions may pass through an emission light filter 26 disposed in the light path between the tissue specimen and the light detector/camera 28A. In those system 20 embodiments that include an emission filter assembly 34, the system controller 32 may control the emission filter system 34 to position a desired one of the emission light filters 26 in the light path between the tissue specimen and the light detector/camera 28A, e.g., the desired emission light filter 26 may be chosen based on the excitation light wavelength used to produce the light emission.

The light detector/camera 28A detecting the fluorescent light emissions is configured to produce signals representative of the fluorescently emitted light, and the location of the same, based on the LED producing the excitation light. The system controller 32 utilizes the aforesaid signals to interpret the various regions of the tissue specimen, e.g., to determine the type of tissue present within the respective regions directly (i.e., via intrinsic fluorescent emissions) or indirectly (e.g., via an extrinsic agent that drawn to tissue based on pH environment contiguous with the respective tissue region). The tissue analysis performed within system controller 32 based on the fluorescent emissions may be used by the system controller 32 to determine the presence or absence of “suspect” tissue regions (i.e., regions where the fluorescent emissions indicate the presence of cancerous tissue or abnormal tissue). If suspect tissue regions are identified, the location of those suspect tissue regions is also identified.

The operation of the present disclosure system 20 embodiment diagrammatically shown in FIG. 1 is substantially like that described above for the system diagrammatically shown in FIG. 2, except that the excised tissue sample may be interrogated by a single excitation light 22 source that is configured to produce both fluorescence emission and Raman scattering, and a single light detector 28 may be used.

Techniques utilizing autofluorescence spectroscopy to differentiate between normal tissue and cancerous tissue are known, and the present disclosure is not limited to using any such technique. However, cancer margin detection may be compromised when the detection technique lacks specificity, and currently available autofluorescence techniques have not thus far demonstrated sufficient specificity to be effective in cancer margin detection. In fact, it has been reported that autofluorescence spectroscopy techniques have lower sensitivities and specificities than that obtainable using Raman spectroscopy. [12]-[16]

In the event a suspect tissue region is determined based on the fluorescence emissions produced from the excised tissue sample, the present disclosure system 20 may then utilize Raman scattering emissions from the identified suspect tissue region to further investigate the suspect tissue region for the presence or absence of cancerous tissue. The Raman scattering emissions can be used to produce information relating to the presence or absence of cancerous tissue with greater specificity and sensitivity than is possible with currently known fluorescence spectroscopy techniques. The present disclosure is not limited to any particular Raman spectroscopy technique for analyzing a tissue sample. Non-limiting examples of acceptable Raman spectroscopy techniques include PCT Publication No. WO 2020/160462 and PCT Application No. PCT/US2021/016090, both of which are incorporated by reference herein in their entirety.

Hence it can be seen from the above that the present disclosure leverages the speed of autofluorescence spectroscopy to determine the presence or absence of any suspect tissue regions on an excised tissue sample, and leverages Raman spectroscopy (a slower process than autofluorescence) to examine those suspect tissue regions to produce tumor margin information with greater specificity. In this manner, the present disclosure system 20 and method can greatly enhance the tumor removal process, and in particular the processes for determining whether all cancerous tissue has been removed and decrease the possibility of subsequent related actions; e.g., follow up surgery to remove residual cancerous tissue.

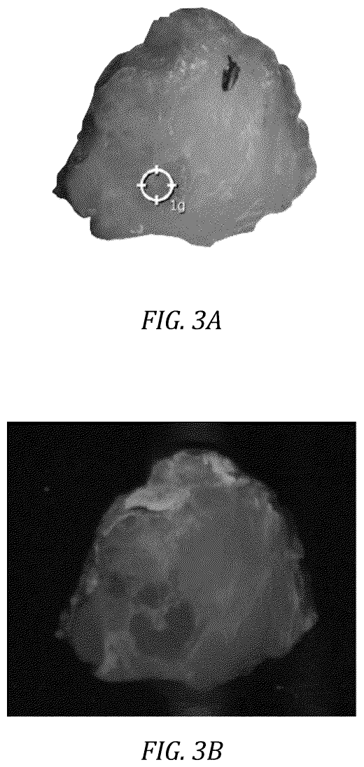

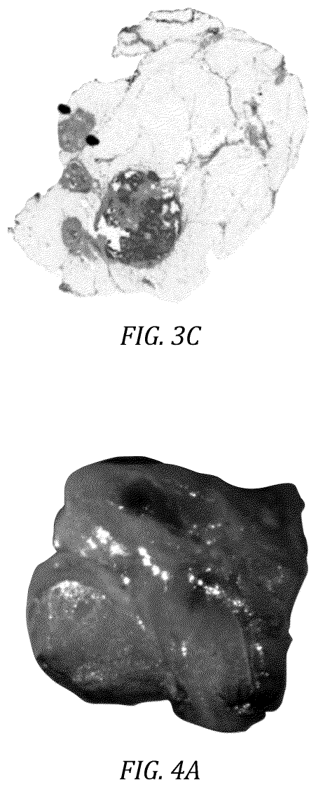

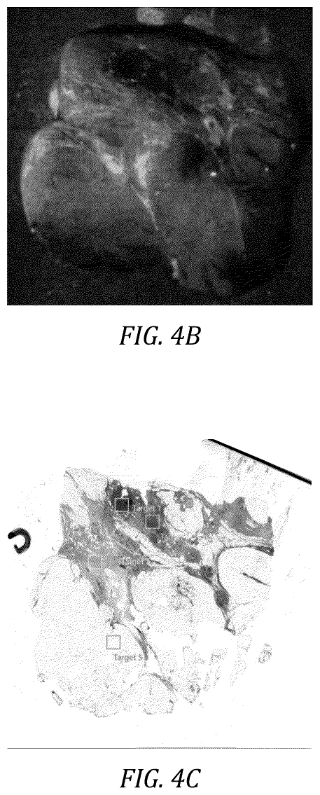

FIGS. 3A-4C illustrate advantages of the present disclosures. FIG. 3A is an image of an excised tissue sample shown in white light. As can be seen in FIG. 3A, the image itself does not overtly reveal any suspect tissue regions. FIG. 3B is an image of the same excised tissue sample, now imaged using autofluorescence spectroscopy with a UV excitation wavelength. Several different tissue regions are highlighted within the FIG. 3B image, indicating differences in tissue regions and/or regions having an acidic microenvironment (i.e., a flag for the presence of cancerous tissue cells). FIG. 3C is an image of the same excised tissue sample subject to an H&E stain. A tissue suspect region is evident in the frontal portion of the excised tissue sample. In similar fashion, FIGS. 4A-4C show images of a particular excised tissue sample. The image shown in FIG. 4A itself does not overtly reveal any suspect tissue regions. The image shown in FIG. 4B (imaged using autofluorescence spectroscopy with a UV excitation wavelength) reveals differences between tissue regions. The image shown in FIG. 4C has been subjected to an H&E stain. Based on the image of the sample with H&E stain shown in FIG. 4C, there does not appear to be any tissue suspect region. Nevertheless, under the present disclosure any suspect tissue region can be evaluated with substantially more sensitivity and specificity than is possible using autofluorescence techniques alone. By using autofluorescence to identify suspect tissue regions and their locations for further investigation via Raman spectroscopy, the present disclosure provides a considerable improvement in speed and effectiveness over presently available techniques.

In addition, the multispectral images produced using the present disclosure can provide improved metabolic insights. Ratiometric analyses using data produced under the present disclosure may be used to bring out better contrast and compare the concentrations and location of native tissue contrasts such as collagen, tryptophan etc. Also, image processing methods based on machine learning and artificial intelligence could be employed to automatically detect and locate suspicious tissue areas on the tissue sample.

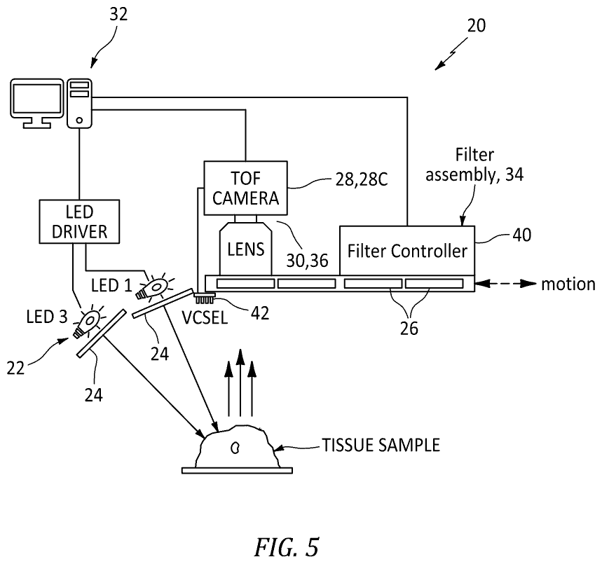

In some embodiments of the present disclosure, an alternative embodiment of the present disclosure may be configured to produce a three-dimensional (3D) map of an excised tissue sample. In this embodiment the system light detector includes a time of flight (TOF) camera 28C as shown in FIG. 5. This system 20 embodiment may facilitate an improved rendering of the excised tissue sample (i.e., the tumor) and may provide desirable feedback to a surgeon. A TOF camera 28C utilizes a charge collection for each pixel in an array that can be switched between two collection devices and read independently. The TOF camera 28C measures time of flight by modulating a source directed at a scene and modulating the per pixel charge collection at the same frequency. The difference in collected charge between the two collection devices for each pixel is a measure of the distance between the source and the scene for that pixel. In these embodiments and for the purpose of making a 3D map of a tumor surface, the at least one light source may include a vertical cavity surface emitting laser (VCSEL) 42 as shown in FIG. 5.

While the principles of the disclosure have been described above in connection with specific apparatuses and methods, it is to be clearly understood that this description is made only by way of example and not as limitation on the scope of the disclosure. Specific details are given in the above description to provide a thorough understanding of the embodiments. However, it is understood that the embodiments may be practiced without these specific details.

It is noted that the embodiments may be described as a process which is depicted as a flowchart, a flow diagram, a block diagram, etc. Although any one of these structures may describe the operations as a sequential process, many of the operations can be performed in parallel or concurrently. In addition, the order of the operations may be rearranged. A process may correspond to a method, a function, a procedure, a subroutine, a subprogram, etc.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements unless the context clearly indicates otherwise. As used herein, “comprises” means “includes.” Thus, “comprising A or B,” means “including A or B, or A and B,” without excluding additional elements.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. Any reference to attached, fixed, connected or the like may include permanent, removable, temporary, partial, full and/or any other possible attachment option.

No element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.” As used herein, the terms “comprise”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements may be described as individual units and shown as independent of one another to facilitate the description. In alternative embodiments, such elements may be configured as combined elements.

### REFERENCES

The following references are each hereby incorporated by reference in their entirety.
1. Nguyen and Tsien, “Fluorescence-guided surgery with live molecular navigation a new cutting edge”, Nat Rev Cancer, 13(9), pp. 653-662, 2013.
2. Tummers, et al., “Real-time intraoperative detection of breast cancer using near-infrared fluorescence imaging and methylene blue”, Eur J Surg Oncol., 40(7), 850-858, 2014.
3. Dahr et al., “A diffuse reflectance spectral imaging system for tumor margin assessment using custom annular photodiode arrays”, Biomedical Optics Express, 3, (12), 2012.
4. Harmsen et al., “Cancer imaging using surface-enhanced resonance Raman scattering nanoparticles”, Nat Protoc; 11(4): 664-87, 2016
5. Pence I., Mahadevan-Jansen A., “Clinical instrumentation and applications of Raman spectroscopy”, Chem Soc Rev.; 45 (7):1958-1979, 2016.
6. Ifa and Eberlin, “Ambient Ionization Mass Spectrometry for Cancer Diagnosis and Surgical Margin Evaluation”, Clin Chem.; 62(1), 2016.
7. Tseng et al., In Vivo Fluorescent Labeling of Tumor Cells with the HaloTag® Technology, Current Chemical Genomics, 6, (Suppl 1-M6) pp. 48-54, 2012
8. Zhao et al., “A transistor-like pH nanoprobe for tumor detection and image-guided surgery”, Nature Biomedical Engineering, 1, 0006 (2016).
9. Zhou et al., “Multicolored pH-tunable and activatable fluorescence nanoplatform responsive to physiologic pH stimuli,” Journal of the American Chemical Society, 134:7803-7811, 2012
10. Korzeniowska et al., Intracellular pH-sensing using core/shell silica nanoparticles, J Biomed Nanotechnol., 10(7), pp. 1336-45, 2014
11. Tan et al., “Induction of pH sensitivity on the fluorescence lifetime of quantum dots by NIR fluorescent dyes”, J. Am. Chem. Soc., 134, 4545-4548 (2012)
12. Volynskaya et al., “Diagnosing breast cancer using diffuse reflectance spectroscopy and intrinsic fluorescence spectroscopy”, Journal of Biomedical Optics 13, 2, 024012, (2008)
13. Nguyen et al., “Near-infrared autofluorescence spectroscopy of in vivo soft tissue sarcomas”, Opt Lett. 2015 Dec. 1; 40(23): 5498-5501
14. Nguyen et al., “Development of a modular fluorescence overlay tissue imaging system for wide-field intraoperative surgical guidance”, J Med Imaging, 2018 5(2):021220
15. Changfang Zhu et al., “Diagnosis of breast cancer using fluorescence and diffuse reflectance spectroscopy: a Monte-Carlo-model-based approach”, J Biomed Opt. 2008; 13 (3):

1. A method of analyzing a resected tissue sample, comprising:
using an imaging system to image a resected tissue sample with excitation light configured to produce fluorescent emissions from the tissue sample, the imaging producing signals representative of the fluorescent emissions from the tissue sample;
determining a presence or an absence of at least one suspect tissue region on the tissue sample;
determining a spatial location of said at least one suspect tissue region determined to be present on the tissue sample;
using the imaging system to image the determined suspect tissue region at the determined spatial location with excitation light configured to produce Raman scattering from the tissue sample, the imaging producing signals representative of the Raman scattering from tissue sample; and
analyzing the determined suspect tissue region using the signals representative of the Raman scattering from the tissue sample.

2. The method of claim 1, wherein the step of using said imaging system to image said resected tissue sample with excitation light configured to produce fluorescent emissions from the tissue sample utilizes a first excitation light source; and
wherein the step of using said imaging system to image said resected tissue sample with excitation light configured to produce Raman scattering from the tissue sample utilizes the first excitation light source.

3. The method of claim 2, wherein the step of using said imaging system to image said resected tissue sample with excitation light to produce fluorescent emissions from the tissue sample and the step of using said imaging system to image said resected tissue sample with excitation light to produce Raman scattering from the tissue sample, include using a light detector to detect both the fluorescent emissions from the tissue sample and the Raman scattering from the tissue sample.

4. The method of claim 3, wherein the step of using said imaging system to image said resected tissue sample with excitation light configured to produce Raman scattering from the tissue sample utilizes a Raman system to process the Raman scattering from the tissue prior to the light detector detecting the Raman scattering from the tissue sample.

5. The method of claim 3, wherein the first excitation light source is a laser.

6. The method of claim 3, wherein the first excitation light source is a laser that produces said excitation light at about 265 nm.

7. The method of claim 2, wherein the first excitation light source is a time-of-flight camera, and the method includes producing a three-dimensional surface map of at least a portion of the resected tissue sample.

8. The method of claim 1, wherein the step of using said imaging system to image said resected tissue sample with excitation light configured to produce fluorescent emissions from the tissue sample utilizes a first excitation light source; and
wherein the step of using said imaging system to image said resected tissue sample with excitation light configured to produce Raman scattering from the tissue sample utilizes a second excitation light source; and
wherein the first excitation light source and the second excitation light source are different types of light source.

9. The method of claim 8, wherein the first excitation light source is an LED, and the second excitation light source is a laser.

10. The method of claim 8, wherein the step of using said imaging system to image said resected tissue sample with excitation light to produce fluorescent emissions from the tissue sample utilizes a first light detector to detect the fluorescent emissions from the tissue sample, and the step of using said imaging system to image said resected tissue sample with excitation light to produce Raman scattering from the tissue sample utilizes a second light detector to detect the Raman scattering from the tissue sample.

11. The method of claim 10, wherein the step of using said imaging system to image said resected tissue sample with excitation light configured to produce Raman scattering from the tissue sample utilizes a spectrometer to process the Raman scattering from the tissue prior to the second light detector detecting the Raman scattering from the tissue sample.

12. A system for analyzing a resected tissue sample, comprising:
at least one excitation light source configured to produce excitation light;
at least one light detector;
a Raman system; and
a system controller in communication with the at least one light source, the at least one light detector, the Raman system, and a non-transitory memory storing instructions, which instructions when executed cause the controller to:
control the at least one excitation light source to interrogate a resected tissue sample with said excitation light to produce fluorescent emissions from the tissue sample;
control the at least one light detector to detect the fluorescence emissions from the tissue sample and produce first signals representative of the detected fluorescence emission;
determine a presence or an absence of at least one suspect tissue region on the tissue sample using the first signals representative of the detected fluorescence emission;
determine a spatial location of said at least one suspect tissue region determined to be present on the tissue sample using the first signals representative of the detected fluorescence emission;
control the at least one excitation light source to interrogate the determined suspect tissue region at the determined spatial location with said excitation light to produce Raman scattering from the tissue sample;
control the at least one light detector to detect the Raman scattering from the tissue sample and produce second signals representative of the detected Raman scattering; and
analyze the determined suspect tissue region using the second signals representative of the Raman scattering from the tissue sample.

13. The system of claim 12, wherein the instructions when executed cause the controller to control the same at least one excitation light source to interrogate the resected tissue sample with said excitation light to produce said fluorescent emissions from the tissue sample, and to interrogate the resected tissue sample with excitation light configured to produce said Raman scattering from the tissue sample.

14. The system of claim 13, wherein the instructions when executed cause the controller to control the same at least one light detector to detect the fluorescence emissions from the tissue sample and produce said first signals representative of the detected fluorescence emission and to detect the Raman scattering from the tissue sample and produce said second signals representative of the detected Raman scattering.

15. The system of claim 14, wherein the instructions when executed cause the controller to control the Raman system to process the Raman scattering from the tissue prior to the at least one light detector detecting the Raman scattering from the tissue sample.

16. The system of claim 12, wherein the at least one excitation light source is a laser.

17. The system of claim 12, wherein the at least one excitation light source is a laser that produces said excitation light at about 265 nm.

18. The system of claim 12, wherein the at least one excitation light source includes a first excitation light source and a second excitation light source; and
wherein the instructions when executed cause the controller to control the first excitation light source to interrogate the resected tissue sample with said excitation light to produce said fluorescent emissions from the tissue sample; and
wherein the instructions when executed cause the controller to control the second excitation light source to interrogate the resected tissue sample with said excitation light to produce said Raman scattering emissions from the tissue sample; and
wherein the first excitation light source and the second excitation light source are different types of light source.

19. The system of claim 18, wherein the first excitation light source is an LED, and the second excitation light source is a laser.

20. The system of claim 12, wherein the at least one light detector includes a first light detector and a second light detector; and
wherein the instructions when executed cause the controller to control the first light detector to detect the fluorescent emissions from the tissue sample; and
wherein the instructions when executed cause the controller to control the second light detector to detect the Raman scattering emissions from the tissue sample.

21. The system of claim 20, wherein the Raman system includes a spectrometer, and the wherein the instructions when executed cause the controller to control the spectrometer to process the Raman scattering from the tissue prior to the second light detector detecting the Raman scattering from the tissue sample.
